(19) United States
(12) Patent Application Publication
(10) Pub. No.: US 2014/0315992 A1
(43) Pub. Date: Oct. 23, 2014

(54) Genetic Alterations Associated with Autism and the Autistic Phenotype and Methods of Use Thereof for the Diagnosis and Treatment of Autism

(71) Applicants: Hakon Hakonarson, Malvern, PA (US); Dexter Hadley, Philadelphia, PA (US); Zhi-Liang Wu, North Potomac, MD (US); Joseph Glessner, Mullica Hill, NJ (US)

(72) Inventors: Hakon Hakonarson, Malvern, PA (US); Dexter Hadley, Philadelphia, PA (US); Zhi-Liang Wu, North Potomac, MD (US); Joseph Glessner, Mullica Hill, NJ (US)

(73) Assignee: The Children's Hospital of Philadelphia, Philadelphia, PA (US)

(21) Appl. No.: 14/131,359

(22) PCT Filed: Jul. 9, 2012
(86) PCT No.: PCT/US12/45959

§ 371 Date: Jul. 10, 2014

Related U.S. Application Data
(60) Provisional application No. 61/646,971, filed on May 15, 2012.
(60) Provisional application No. 61/505,352, filed on Jul. 7, 2011.

(51) Int. Cl. C12Q 1/68

(52) U.S. Cl. CPC C12Q 1/6883
USPC 514/455; 435/6.11

(57) ABSTRACT

Compositions and methods for the detection and treatment of autism and autistic spectrum disorder are provided.

This application claims priority to U.S. Provisional Application Nos. 61/505,352 and 61/646,971 filed Jul. 7, 2011 and May 15, 2012 respectively, the entire contents of each being incorporated by reference herein as though set forth in full.

### FIELD OF THE INVENTION

This invention relates to the fields of genetics and the diagnosis and treatment of autism and autism spectrum disorders.

### BACKGROUND OF THE INVENTION

Several publications and patent documents are cited throughout the specification in order to describe the state of the art to which this invention pertains. Each of these citations is incorporated herein by reference as though set forth in full.

Autism (MIM [209850]) is a severe and relatively common neuropsychiatric disorder characterized by abnormalities in social behavior and communication skills, with tendencies towards patterns of abnormal repetitive movements and other behavior disturbances. Current prevalence estimates are ˜0.2% of the population for autism and 0.9% of the population for ASDs (MMWR Surveill Summ. 2009). Globally, males are affected four times as often as females². As such, autism poses a major public health concern of unknown cause that extends into adulthood and places an immense economic burden on society. The most prominent features of autism are social and communication deficits. The former are manifested in reduced sociability (reduced tendency to seek or pay attention to social interactions), a lack of awareness of social rules, difficulties in social imitation and symbolic play, impairments in giving and seeking comfort and forming social relationships with other individuals, failure to use nonverbal communication such as eye contact, deficits in perception of others' mental and emotional states, lack of reciprocity, and failure to share experience with others. Communication deficits are manifested as a delay in or lack of language, impaired ability to initiate or sustain a conversation with others, and stereotyped or repetitive use of language. Autistic children have been shown to engage in free play much less frequently and at a much lower developmental level than peers of similar intellectual abilities. Markers of social deficits in affected children appear as early as 12-18 months of age, suggesting that autism is a neurodevelopmental disorder. It has been suggested that autism originates in developmental failure of neural systems governing social and emotional functioning. Although social and cognitive development are highly correlated in the general population, the degree of social impairment does not correlate well with IQ in individuals with autism. The opposite is seen in Down's syndrome and Williams syndrome, where social development is superior to cognitive function. Both examples point to a complex source of sociability.

The etiology of the most common forms of autism is still unknown. In the first description of the disease in 1943, Kanner suggested an influence of child-rearing practices on the development of autism, after observing similar traits in parents of the affected children. While experimental data fail to support several environmental hypotheses, there has been growing evidence for a strong genetic influence on this disorder. The rate of autism in siblings of affected individuals was shown to be 8.6%, a 215 fold increase over the general population (Ritvo et al. (1989) Am J Psychiatry 146(8):1032-6). Twin studies have demonstrated significant differences in monozygotic and dizygotic twin concordance rates, the former concordant in 60% of twin pairs, with most of the non-autistic monozygotic co-twins displaying milder related social and communicative abnormalities. Social, language and cognitive difficulties have also been found among relatives of autistic individuals in comparison to the relatives of controls. The heritability of autism has been estimated to be >90%.

The genetic basis of autism has been extensively studied in the past decade using three complementary approaches: cytogenetic studies; linkage analysis, and candidate gene analysis see for a review (Freitag, C. M. et al., (2010) Eur Child Adolesc Psychiatry 19(3):169-78; Vorstman et al., (2006) Mol. Psychiatry. 11:18-28; Veenstra-VanderWeele and Cook, (2004) Mol. Psychiatry. 9: 819-32). Searches for chromosomal abnormalities in autism have revealed terminal and interstitial deletions, balanced and unbalanced translocations, and inversions on a large number of chromosomes, with abnormalities on chromosomes 15, 7, and X being most frequently reported. The importance of the regions indicated by cytogenetic studies was evaluated by several whole genome screens in the multiplex autistic families (International Molecular Genetic Study of Autism Consortium, 1998). Strong and concordant evidence for the presence of an autism susceptibility locus was obtained for chromosome 7q; moderate evidence was obtained for loci on chromosomes 15q, 16p, 19p, and 2q; and the majority of the studies find no support for linkage to the X chromosome (Lamb et al, (2005) Med. Genet. 42: 132-137; Lord et al, (2000) Autism Dev Disord. 30:205-223; Muhle et al., (2004) Pediatrics 113(5): e472-86). The AGRE sample provided the strongest evidence for loci on 17q and 5p (Yonan et al., (2003) Am J Hum Genet. 73:886-97). Numerous candidate gene studies in autism have focused on a few major candidates with respect to their location or function (reviewed in Veenstra-VanderWeele et al 2004, supra). Jamain et al., ((2003) Nat. Genet. 34:27-9), reported rare nonsynonymous mutations in the X-linked genes encoding neuroligins, specifically NLGN3 and NLGN4, in linkage regions associated with ASD. Other evidence for a genetic basis of autistic endophenotypes comes from the study of disorders that share phenotypic features that overlap with autism such as Fragile X and Rett syndrome.

Many emerging theories of autism focus on changes in neuronal connectivity as the potential underlying cause of these disorders. Imaging studies reveal changes in local and global connectivity (Just et al., (2004) Brain 127: 1811-1821; Herbert et al., (2005) Ann Neurol 55(4): 530-40) and developmental studies of activity-dependent cortical development suggest that autism might result from an imbalance of inhibitory and excitatory synaptic connections during development (Rubenstein and Merzenich, (2004; Genes Brain Behav 2(5): 255-67). The fundamental unit of neuronal connectivity is the synapse; thus, if autism is a disorder of neuronal connectivity, then it can likely be understood in neuronal terms as a disorder of synaptic connections. Indeed, genetic studies reveal that mutations in key proteins involved in synaptic development and plasticity, such as neuroligins, FMRP and MeCP2 are found in individuals with autism and in two forms of mental retardation with autistic features, specifically Fragile-X and Rett's syndrome (Jamain et al, 2003, supra, O'Donnell and Warren, (2002) Arum Rev Neurosci 25: 315-38). Thus the pursuit of linkage between genetic anomalies and (endo)phenotypes at the neuronal level appears both warranted and fruitful. Furthermore, such neuronal connectivity anomalies, revealed, for example, by direct white matter tractography, or by observable delays in characteristic electrical activity, can be directly linked to behavioral and clinical manifestations of ASD, allowing these neuron-level phenotypes to be interpreted as neural correlates of behavior.

Overall, the linkage analysis studies conducted to date and discussed above have achieved only limited success in identifying genetic determinants of autism due to numerous reasons, among others the generic problem that the linkage analysis approach is generally poor in identifying common genetic variants that have modest effects (Hirschhorn and Daly, (2005) Nat Rev Genet. 6(2): 95-108). This problem is highlighted in autism, a spectrum disorder wherein the varied phenotypes are determined by the net result of interactions between multiple genetic and environmental factors and, in which, any particular genetic variant that is identified is likely to contribute little to the overall risk for disease.

In one of the first studies to report an association of de novo copy number variations (CNVs) with autism (Science (2007) Apr. 20; 316(5823):445-9), Sebat and colleagues suggest that CNVs may underlie certain cases of the disease. Indeed, the importance of their findings have been recapitulated in more recent work (Pinto et al, Nature. 2010 Jul. 15; 466(7304):368-72.; Glessner et al Nature. 2009 May 28; 459(7246):569-73) suggesting that CNVs may at least account for a small percentage of the genetic variation of the ASDs. However, these genetic defects are rare and collectively only explain a small proportion of the genetic risk for autism, thus suggesting the existence of additional genetic loci but with unknown frequency and effect size.

### SUMMARY OF THE INVENTION

The present inventors have performed genome wide association study on several large patient cohorts and have successfully identified a number of target genes harboring copy number variations associated with autism and ASD. Thus, in accordance with the present invention, a method for detecting a propensity for developing autism or autistic spectrum disorder is provided. An exemplary method comprises obtaining a sample from a patient and testing the sample for the presence or absence of at least one deletion containing CNV in a target polynucleotide, wherein if the CNV is present, the patient has an increased risk for developing autism and/or autistic spectrum disorder. In a preferred embodiment, the deletion containing CNV is selected from the group of CNVs provided in Table II. In another embodiment, the step of detecting the presence of said CNV further comprises performing a process selected from the group consisting of detection of specific hybridization, measurement of allele size, restriction fragment length polymorphism analysis, allele-specific hybridization analysis, single base primer extension reaction, and sequencing of an amplified polynucleotide.

In another aspect, the present invention provides a method for identifying agents which alter neuronal signaling and/or morphology. An exemplary method entails providing cells expressing at least one CNV listed in Table 2 and cells which express the cognate wild type sequences corresponding to the CNV containing sequence, contacting both cell types with a test agent and analyzing whether the agent alters neuronal signaling and/or morphology of cells comprising the CNV relative to those which lack the genetic alteration, thereby identifying agents which alter neuronal signaling and morphology in CNV containing cells.

Also provided is a method of treating autism or ASD in a human subject determined to have at least copy number variation (CNV) associated with an autistic or ASD phenotype, said at least one CNV being selected from the group consisting of CNVs set out in Table 2, the method comprising administering to said human subject a therapeutically effective amount of at least one agent which is known to be efficacious in the signaling pathway adversely affected by the presence of said CNV. In a preferred embodiment, patients are tested for the presence or absence of at least one CNV containing gene is selected from the group consisting of ATP 10A, GABRA5, GABRB3, GABRG3, GGTLC2, HBII-52-45, HBII-52-46, IPW, LOC648691, LOC96610, MAGEL2, MIR650, MKRN3, NCRNA00221, NDN, OCA2, OR4S2, PAR-SN, PAR1, PARS, POM121L1P, PRAME, SNORD107, SNORD108, SNORD109A, SNORD109B, SNORD115-11, SNORD115-29, SNORD115-36, SNORD115-43, SNORD115-44, SNORD115-48, SNORD64, SNRPN, SNURF, UBE3A, ZNF280A, ZNF280B. In yet another embodiment, the CNV is determined to reside in a gene important for GABA signaling and the agent is listed in Table 3 or Table 4. In a particularly preferred embodiment, the CNV alters GABA signaling and the agent is topiramide.

### BRIEF DESCRIPTION OF THE DRAWINGS

FIG. 1 shows the design of the present study. In this two-stage design, 2076 cases vs 4754 controls were used in the discovery cohort (Stage 1), and 1159 cases vs 2546 controls were used for a replication cohort (Stage 2). All samples used passed minimal quality control metrics, but the default quality calls of PennCNV were used to discriminate the discovery cohort (best quality) from the replication cohort (lesser quality.)

FIG. 2 is a graph showing the classes of gene pathways, which are disrupted by the ASD-related CNVRs disclosed herein. All genes with exons disrupted by replicated CNVRs were submitted to Ingenuity to ascertain significance of pathway enrichment.

FIG. 3 is a schematic of a first-degree interactome of the GABAR-A family highlighting copy number defects enriched in cases (red) vs controls (blue).

FIG. 4: Test and treat model for targeting therapeutics to specific pathways defective in disease. The generic test and treat model is shown in black where a molecular diagnostic is used to genetically define a population with defective pathways that are likely to benefit from a targeted intervention. Examples of trastuzumab as a targeted intervention for HER2 specific breast cancer is shown in blue as well as an extrapolation of behavioral programs and novel therapeutics that are being developed to target ASDs due to defective GABAR-A pathways in red.

### DETAILED DESCRIPTION OF THE INVENTION

Epidemiologic studies have convincingly implicated genetic factors in the pathogenesis of autism, a common neuropsychiatric disorder in children, which presents with variable phenotype expression that extends into adulthood. Several genetic determinants have already been reported to be associated with ASD, including many rare de novo copy number variants (CNVs) that harbor small genomic deletions and insertions. These genetic alterations may account for a small subset of the phenotypic manifestation of the disease. Implicated genomic regions appear to be highly heterogeneous with variations reported in several genes, including NRXN1, NLGN3, SHANK3 and AU7S2 to date.

Predicting an individual's genomic risk for disease can facilitate the development of new interventions and streamline therapeutic approaches. To identify likely functional CNVs, we combined various large cohorts of autistic patients with a large number of neurologically normal controls to analyze over 3K affected cases and 7K controls. In a two-stage genome-wide association design, we uncovered 266 genome-wide statistically significant (combined P<=2.76×10⁻⁸) distinct CNV regions (CNVR).

The 38 genes with exons disrupted by these robust CNVRs are most enriched in gene networks impacting neurological disease, behavior and developmental disorders. GABAR-A receptor signaling was the most significant disrupted canonical pathway in ASD where case-enriched defects in GABRA5, GABRB3, and GABRG3 genes were identified. Moreover, network analysis of the first-degree gene interactome of the GABAR-A receptor family suggests that ASD cases are significantly enriched for such pathway defects (P<=2.1×¹⁰−21, OR=9.9) when compared with neurologically normal controls.

Taken together, the CNVRs we have identified impact multiple novel genes and signaling pathways, including genes involved in GABAR-A signaling, that can provide important targets for therapeutic intervention.

### DEFINITIONS

A “copy number variation (CNV)” refers to the number of copies of a particular gene in the genotype of an individual. CNVs represent a major genetic component of human phenotypic diversity. Susceptibility to genetic disorders is known to be associated not only with single nucleotide polymorphisms (SNP), but also with structural and other genetic variations, including CNVs. A CNV represents a copy number change involving a DNA fragment that is −1 kilobases (kb) or larger (Feuk et al. 2006a). CNVs described herein do not include those variants that arise from the insertion/deletion of transposable elements (e.g., ˜6-kb KpnI repeats) to minimize the complexity of future CNV analyses. The term CNV therefore encompasses previously introduced terms such as large-scale copy number variants (LCVs; Iafrate et al. 2004), copy number polymorphisms (CNPs; Sebat et al. 2004), and intermediate-sized variants (ISVs; Tuzun et al. 2005), but not retroposon insertions.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with autism. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any autism specific marker gene or nucleic acid, but does not hybridize to other nucleotides. Also polynucleotide which “specifically hybridizes” may hybridize only to a neurospecific specific marker, such an autism-specific marker shown in the Table contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5″C+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57″C. The Tₘ of a DNA duplex decreases by 1-1.5″C with every 1% decrease in homology. Thus, targets with greater than about 75% sequence identity would be observed using a hybridization temperature of 42″C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid.

Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC. and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product. Probes and primers having the appropriate sequence homology which specifically hybridized to CNV containing nucleic acids are useful in the detecting the presence of such nucleic acids in biological samples.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the Autism specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the autism specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably an autism specific marker molecule, such as a marker shown in the tables provided below. Samples may include but are not limited to cells, body fluids, including blood, serum, plasma, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP and/or CNV containing nucleic acids described herein or their encoded proteins. Agents are evaluated for potential biological activity by inclusion in screening assays described hereinbelow.

### Methods of Using Autism-Associated CNVS and/or SNPS for Diagnosing a Propensity for the Development of Autism and Autistic Spectrum Disorders

Autism-related-CNV and/or SNP containing nucleic acids, including but not limited to those listed in the Table provided below may be used for a variety of purposes in accordance with the present invention. Autism-associated CNV/SNP containing DNA, RNA, or fragments thereof may be used as probes to detect the presence of and/or expression of autism specific markers. Methods in which autism specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting autism-associated CNVs/SNPs may be conducted on any type of biological sample, including but not limited to body fluids (including blood, urine, serum, gastric lavage), any type of cell (such as brain cells, white blood cells, mononuclear cells) or body tissue. Such detection methods can include for example, southern and northern blotting, RFLP, direct sequencing and PCR amplification followed by hybridization of amplified products to a microarray comprising reference nucleic acid sequences.

From the foregoing discussion, it can be seen that autism-associated CNV/SNP containing nucleic acids, vectors expressing the same, autism CNV/SNP containing marker proteins and anti-Autism specific marker antibodies of the invention can be used to detect autism associated CNVs/SNPs in body tissue, cells, or fluid, and alter autism SNP containing marker protein expression for purposes of assessing the genetic and protein interactions involved in the development of autism.

In most embodiments for screening for autism-associated CNVs/SNPs, the autism-associated CNV/SNP containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art. Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus any of the aforementioned techniques may be used to detect or quantify autism-associated CNV/SNP marker expression and accordingly, diagnose autism or an autism spectrum disorder.

### Kits and Articles of Manufacture

Any of the aforementioned products can be incorporated into a kit which may contain a autism-associated CNV/SNP specific marker polynucleotide or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

### Methods of Using Autism-Associated CNVS/SNPS for Development of Therapeutic Agents

Since the CNVs and SNPs identified herein have been associated with the etiology of autism, methods for identifying agents that modulate the activity of the genes and their encoded products containing such CNVs/SNPs should result in the generation of efficacious therapeutic agents for the treatment of a variety of disorders associated with this condition.

As can be seen from the data provided in Table 1, several chromosomes contain regions which provide suitable targets for the rational design of therapeutic agents which modulate their activity. Small peptide molecules corresponding to these regions may be used to advantage in the design of therapeutic agents which effectively modulate the activity of the encoded proteins.

Molecular modeling should facilitate the identification of specific organic molecules with capacity to bind to the active site of the proteins encoded by the CNV/SNP containing nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered autism associated gene. These host cell lines or cells are defective at the polypeptide level. The host cell lines or cells are grown in the presence of drug compound. The rate of cellular metabolism, alterations in cellular morphology and/or receptor signaling of the host cells is measured to determine if the compound is capable of altering any of these parameters in the defective cells. Host cells contemplated for use in the present invention include but are not limited to bacterial cells, fungal cells, insect cells, mammalian cells, and plant cells. The autism-associated CNV/SNP encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the autism-associated CNVs/SNPs of the present invention or functional fragments thereof provide a system in which to screen potential compounds or agents for the ability to modulate the development of autism. Thus, in one embodiment, the nucleic acid molecules of the invention may be used to create recombinant cell lines for use in assays to identify agents which modulate aspects of cellular metabolism associated with neuronal signaling and neuronal cell communication and structure. Also provided herein are methods to screen for compounds capable of modulating the function of proteins encoded by CNV/SNP containing nucleic acids.

Another approach entails the use of phage display libraries engineered to express fragment of the polypeptides encoded by the CNV/SNP containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the therapeutic.

Thus, one may design drugs which have, e.g., improved polypeptide activity or stability or which act as inhibitors, agonists, antagonists, etc. of polypeptide activity. By virtue of the availability of CNV/SNP containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of autism-associated CNV/SNP containing nucleic acids enables the production of strains of laboratory mice carrying the autism-associated CNVs/SNPs of the invention. Transgenic mice expressing the autism-associated CNV/SNP of the invention provide a model system in which to examine the role of the protein encoded by the SNP containing nucleic acid in the development and progression towards autism. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic and neuronal processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of autism-associated CNV/SNP containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated autism-associated CNV/SNP genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing autism-associated SNP containing nucleic acid as a targeted insertional cassette provides means to detect a successful insertion as visualized, for example, by acquisition of immunoreactivity to an antibody immunologically specific for the polypeptide encoded by autism-associated SNP nucleic acid and, therefore, facilitates screening/selection of ES cells with the desired genotype.

As used herein, a knock-in animal is one in which the endogenous murine gene, for example, has been replaced with human autism-associated CNV/SNP containing gene of the invention. Such knock-in animals provide an ideal model system for studying the development of autism.

As used herein, the expression of a autism-associated CNV/SNP containing nucleic acid, fragment thereof, or an autism-associated CNV/SNP fusion protein can be targeted in a “tissue specific manner” or “cell type specific manner” using a vector in which nucleic acid sequences encoding all or a portion of autism-associated CNV/SNP are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding the autism-associated CNV/SNP of the invention may be operably linked to a variety of different promoter sequences for expression in transgenic animals. Such promoters include, but are not limited to a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; and Glial fibrillar acidic protein (GFAP) promoter for the expression of transgenes in glial cells.

Methods of use for the transgenic mice of the invention are also provided herein. Transgenic mice into which a nucleic acid containing the autism-associated CNV/SNP or its encoded protein have been introduced are useful, for example, to develop screening methods to screen therapeutic agents to identify those capable of modulating the development of autism.

### Pharmaceuticals and Peptide Therapies

The elucidation of the role played by the autism associated CNVs/SNPs described herein in neuronal signaling and brain structure facilitates the development of pharmaceutical compositions useful for treatment and diagnosis of autism. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following materials and methods are provided to facilitate the practice of the present invention.

### Study Design & Quality Control

PennCNV was used to define CNVs across all genotyped samples. To control for potential chip-to-chip bias from the mixed SNP content introduced by genotyping across multiple chips types, only CNV calls from the 550K joint SNPs across the 550K, 610K, 660K, and 1M Illumina chips were considered. Low quality samples were excluded on a per sample basis if:

1. # CNVs>100

2. SD LRR>0.3

3. |GCWF|>0.02

### Statistical Analysis

For each stage of analysis, the genome was segmented into CNV regions (CNVRs) that define unambiguous sets of cases and controls impacted by CNVs which facilitates the immediate identification of “core” CNV genomic regions. These CNVRs were tested for association by Fisher's exact test in a two-stage design with an alpha of P<=0.01 after correcting for multiple tests.

### Network Analysis

Ingenuity pathway analysis was used to look for enrichment in networks and canonical pathways among genes with exons disrupted by replicated CNVRs. Fisher's exact test was used to gauge enrichment of the first order interactome of GABAR family of genes, as well as a test of 1000 random permutations of case/control labels.

The following examples are provided to illustrate certain embodiments of the invention. They are not intended to limit the invention in any way.

### Example I

The ability to quantify individual's genomic risk for disease can facilitate the development of new interventions and improve medical practice. Many rare Copy Number Variants (CNVs) that harbor small genomic deletions and insertions have been described in the autism spectrum disorders (ASD). To identify these likely functional elements, we combined various large cohorts of autistic patients with a large number of neurologically normal controls to analyze over 3K affected cases and 7K controls. In a two-stage genome-wide association design, we uncovered 266 genome-wide statistically significant (combined P<=2.76×10⁻⁸) distinct CNV regions (CNVR).

The 38 genes with exons disrupted by these robust CNVRs are most enriched in gene networks impacting neurological disease, behavior and developmental disorder. GABAR-A receptor signaling was found to be the most significant canonical pathways disrupted in ASD because case-enriched defects in GABRA5, GABRB3, and GABRG3 genes. Moreover, network analysis of the first-degree gene interactome of the GABAR-A receptor family suggests that ASD cases are significantly enriched for pathway defects (P<=2.1×10⁻²¹, OR=9.9) when compared with neurologically normal controls.

Taken together, the CNVRs we have identified impact multiple novel genes and signaling pathways, including genes involved in GABAR-A signaling, that may be important for new therapeutic development.

### Results

In all, 3871 unrelated cases were compared to 7768 controls. Samples were sourced from five independent sites, and were distributed as follows:

TABLE 1

| Cohort | # SNPs measured | #cases | #controls |
|---|---|---|---|
| Site #1 | 550K | 926 | |
| Site #2 | 550K | 1237 | |
| Site #3 | 1M | 799 | |
| Site #4 | 610K + 660K | 266 | |
| Site #5 | 550K + 610K + 660K | 643 | 7768 |
| Total | | 3871 | 7768 |

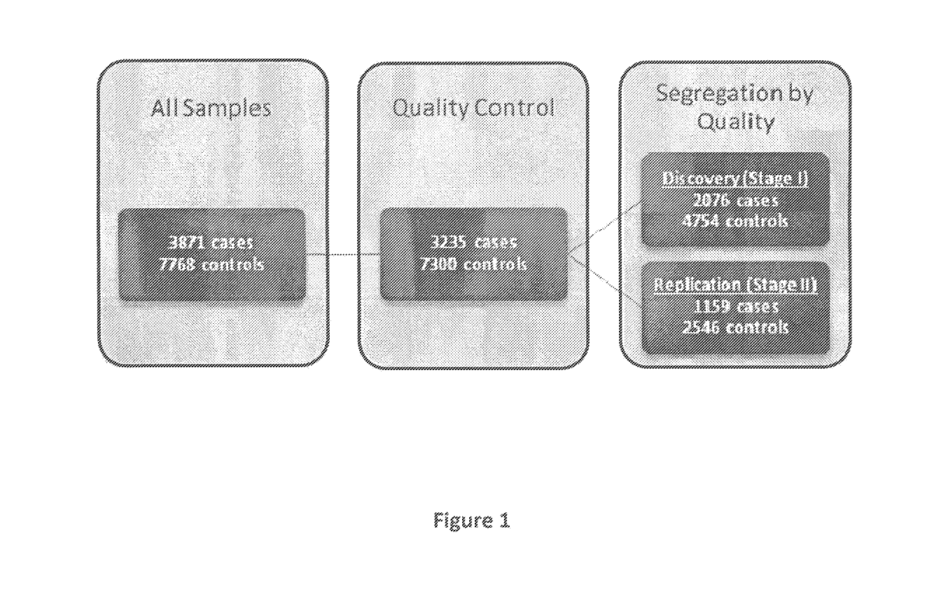

In all, 3225 cases and 7300 controls passed quality control and were used for CNV analysis. These individuals were segregated into a discovery (stage 1) and replication (stage 2) cohort based on the default quality calls of PennCNV. In this two-stage design, 2076 cases vs 4754 controls were used in the discovery cohort, and 1159 cases vs 2546 controls were used for a replication cohort. See FIG. 1.

In the discovery stage, 353 significant CNVRs (nominal P<=1.8×10⁻⁸) were identified after Bonferroni correction for 550K SNPs used for analysis, and 266 significant CNVRs replicated (nominal P<=2.9×10⁻⁵) after correcting for 353 significant discovery regions tested. The most significantly associated CNVRs highlight some attractive and novel candidate genes for ASD.

Most interesting are the 25 duplications unique to cases in GABRB3-GABA-A receptor, beta 3 (P<=1.42×10⁴³, OR=inf). This is an attractive candidate gene as GABA is the main inhibitory neurotransmitter, and it lies within the Prader-Willi/Angelman syndrome critical region (15q11-13), mutations of which have been described in several individuals with autism. Moreover, this was found to be significant across Europeans and African populations (P<=6.44×10⁻⁵ and 1.82×10⁵ respectively); Association between a GABRB3 polymorphism and autism (Buxbaum et al., 2002) as well as GABRA4 & GABRB1 (Collins et al., 2006). Gabrb3 gene deficient mice exhibit impaired social and exploratory behaviors, deficits in non-selective attention and hypoplasia of cerebellar vermal lobules: a potential model of autism spectrum disorder (DeLorey, Sahbaie, Hashemi, Homanics, & Clark, 2008)

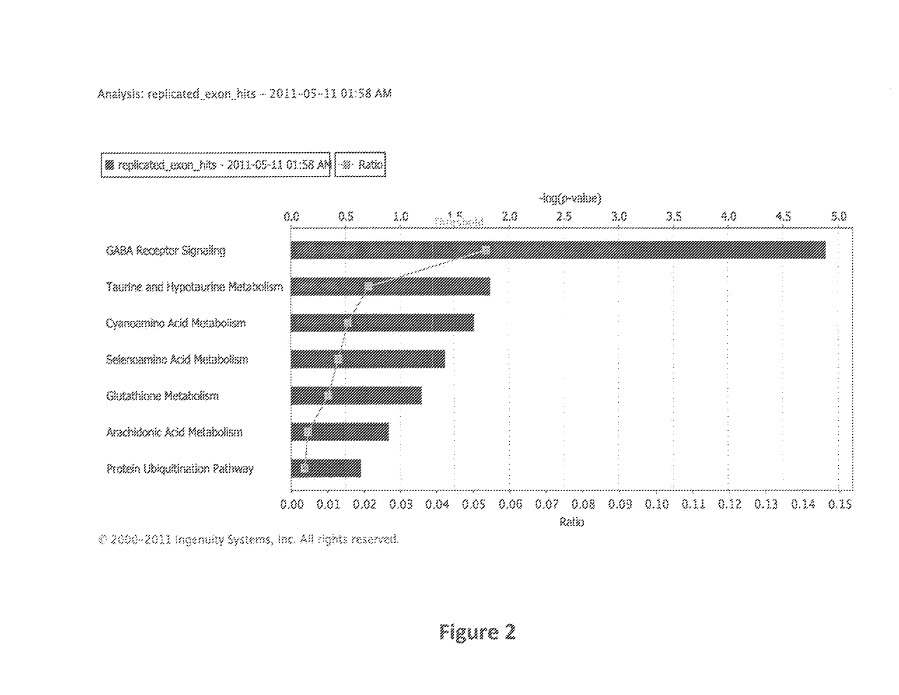

We found 38 genes with exons disrupted by robust CNVRs: ATP10A, GABRA5, GABRB3, GABRG3, GGTLC2, HBII-52-45, HBII-52-46, IPW, LOC648691, LOC96610, MAGEL2, MIR650, MKRN3, NCRNA00221, NDN, OCA2, OR4S2, PAR-SN, PAR1, PARS, POM121L1P, PRAME, SNORD107, SNORD108, SNORD109A, SNORD109B, SNORD115-11, SNORD115-29, SNORD115-36, SNORD115-43, SNORD115-44, SNORD115-48, SNORD64, SNRPN, SNURF, UBE3A, ZNF280A, ZNF280B. These genes are most enriched in gene networks impacting neurological disease, behavior and developmental disorder, and GABAR-A receptor signaling was found to be the most significant canonical pathways disrupted in ASD associated with case-enriched defects in GABRA5, GABRB3, and GABRG3 genes. See FIG. 2 and Table 2.

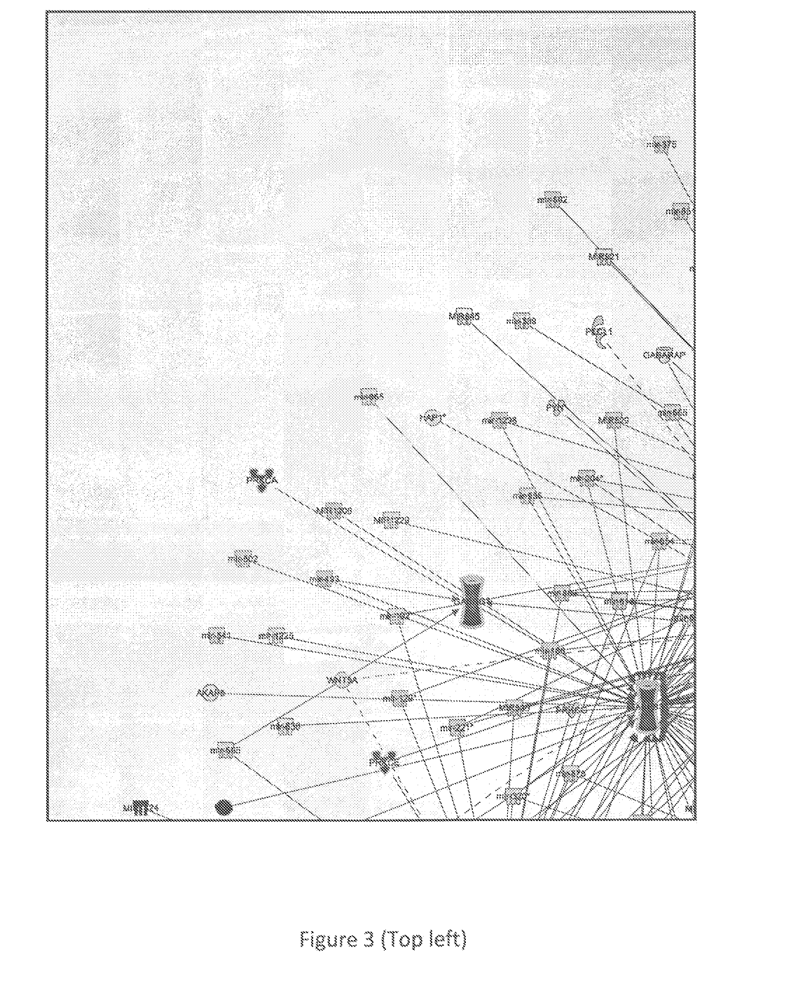

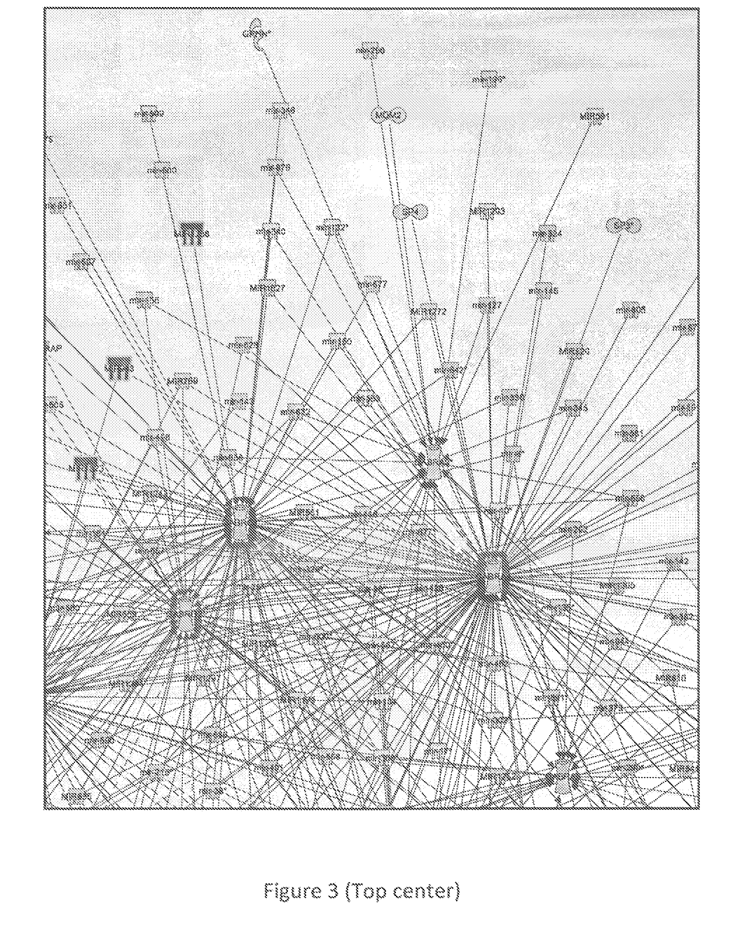

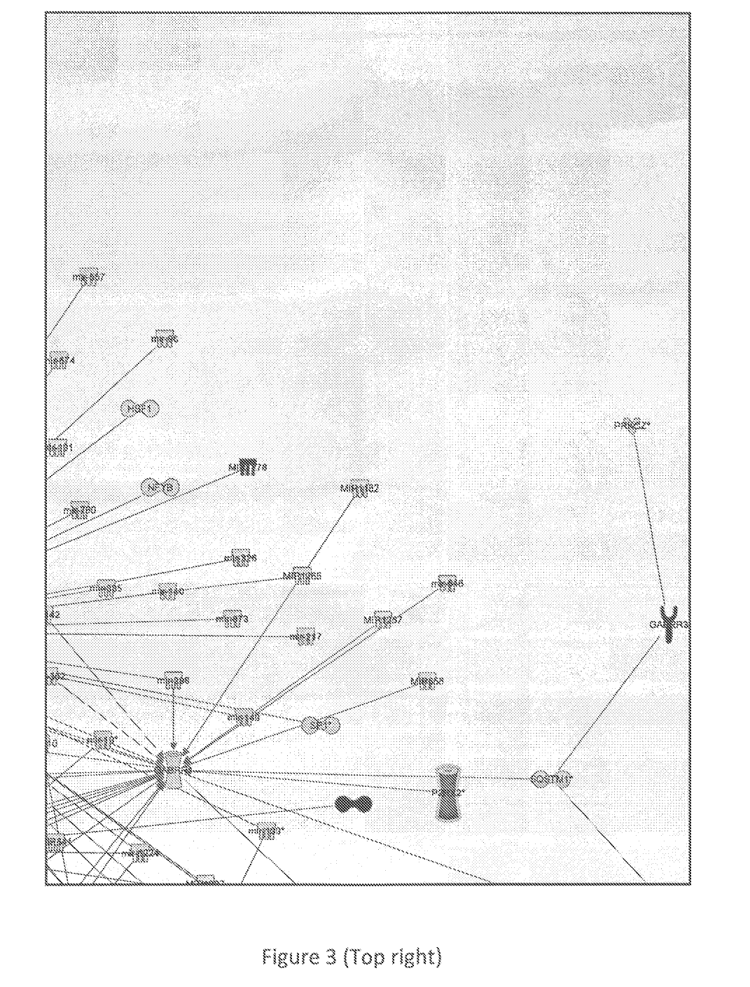

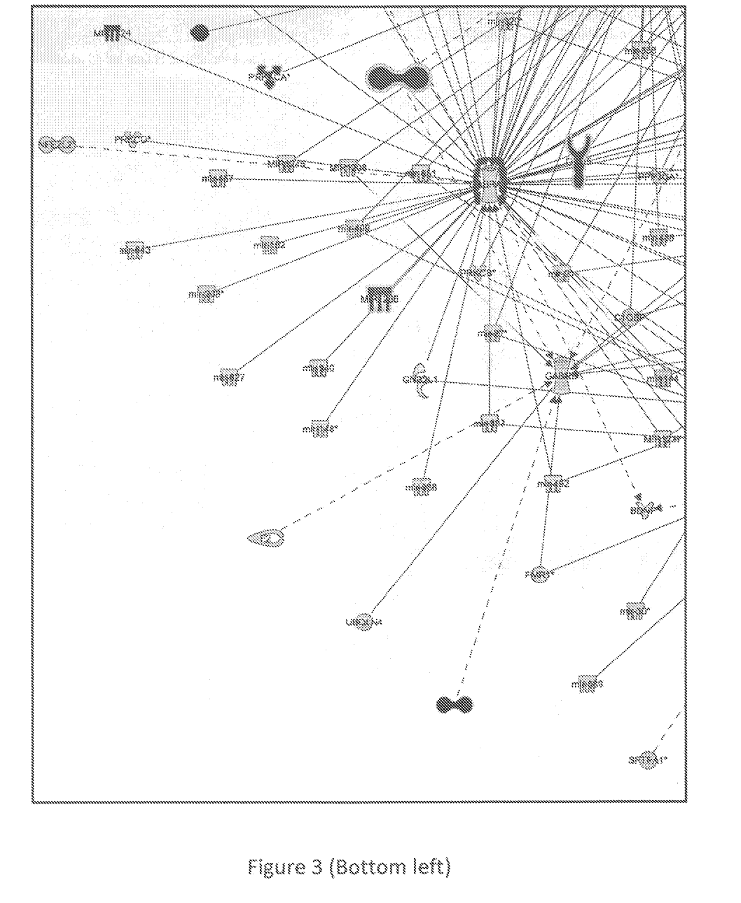

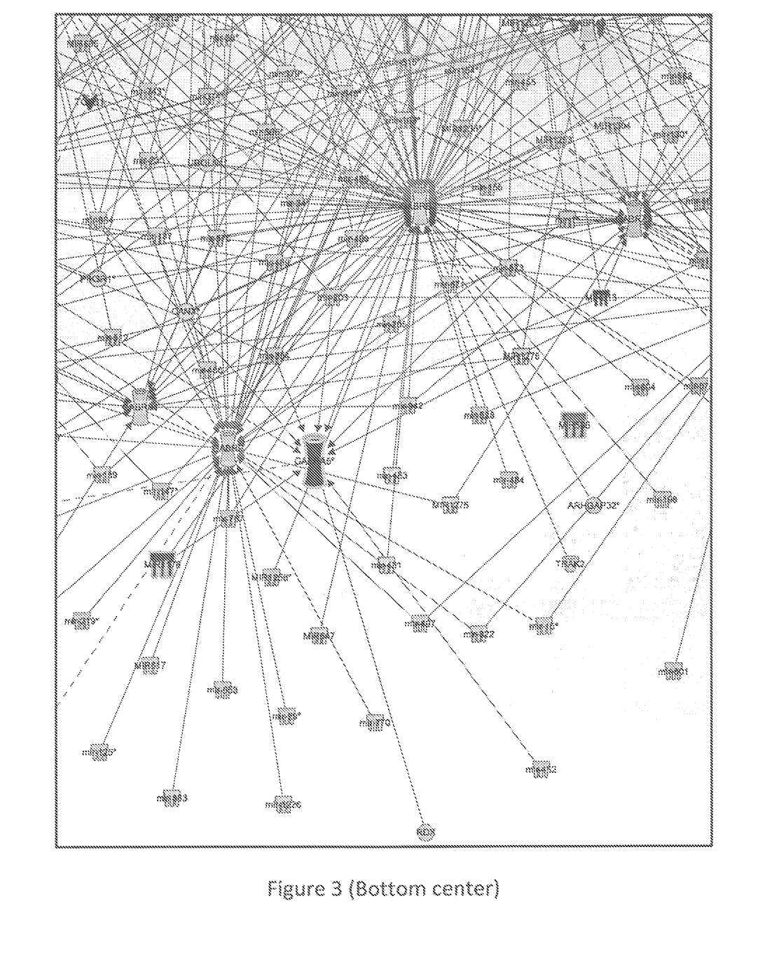

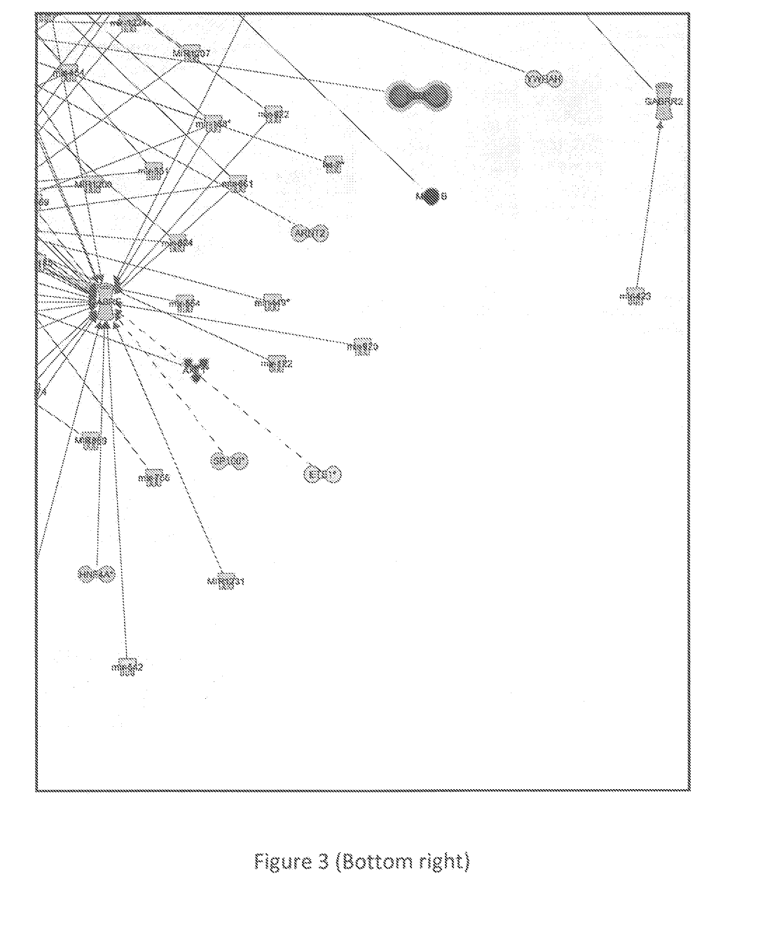

Finally, we defined the first-degree interactome of the GABAR-A family, and a found that ASD cases are significantly enriched for pathway defects in cases when compared with neurologically normal controls. About 3% of cases harbor genetic pathway defects vs 0.03% of controls (P<=2.1×10⁻²¹, OR=9.9), and 17 out of 121 genes enriched in cases (14%) vs 9 out of 121 genes in controls (7%). The network showing genes enriched in cases (red) vs controls (blue) is as shown in FIG. 3.

TABLE 2
The most significant CNVRs disrupting genes

| band | size | numSnp | bestP | bestOr | bestP | discoveryBestP | replicationBestP | delCases | delControls |
|---|---|---|---|---|---|---|---|---|---|
| [symbol] | [symbol] | [symbol] | [symbol] | [symbol] | [symbol] | [symbol] | [symbol] | [symbol] | [symbol] |
| 11q11 | 4405 | 2 | 1.46E−18 | 1.58 | 1.45699E−18 | 2.14E−12 | 1.51E−07 | 776 | 1215 |
| [symbol] | [symbol] | [symbol] | [symbol] | [symbol] | [symbol] | [symbol] | [symbol] | [symbol] | [symbol] |
| 15q11.2 | 102655 | 19 | 8.28E−13 | 59.14 | 8.28009E−13 | 1.50E−08 | 8.74E−06 | 3 | 2 |
| [symbol] | [symbol] | [symbol] | [symbol] | [symbol] | [symbol] | [symbol] | [symbol] | [symbol] | [symbol] |
| 15q11.2 | 65697 | 7 | 2.61E−12 | 56.85 | 2.61199E−12 | 1.50E−08 | 2.81E−05 | 3 | 2 |
| [symbol] | [symbol] | [symbol] | [symbol] | [symbol] | [symbol] | [symbol] | [symbol] | [symbol] | [symbol] |
| 15q11.2 | 141181 | 34 | 2.61E−12 | 56.85 | 2.61199E−12 | 1.50E−08 | 2.81E−05 | 3 | 2 |
| [symbol] | [symbol] | [symbol] | [symbol] | [symbol] | [symbol] | [symbol] | [symbol] | [symbol] | [symbol] |
| 15q11.2 | 29599 | 2 | 2.61E−12 | 56.85 | 2.61199E−12 | 1.50E−08 | 2.81E−05 | 1 | 2 |
| [symbol] | [symbol] | [symbol] | [symbol] | [symbol] | [symbol] | [symbol] | [symbol] | [symbol] | [symbol] |
| 15q12 | 52133 | 28 | 2.61E−12 | 56.85 | 2.61199E−12 | 1.50E−08 | 2.81E−05 | 3 | 2 |
| [symbol] | [symbol] | [symbol] | [symbol] | [symbol] | [symbol] | [symbol] | [symbol] | [symbol] | [symbol] |
| 15q12 | 173668 | 15 | 2.61E−12 | 56.85 | 2.61199E−12 | 1.50E−08 | 2.81E−05 | 3 | 2 |
| [symbol] | [symbol] | [symbol] | [symbol] | [symbol] | [symbol] | [symbol] | [symbol] | [symbol] | [symbol] |
| 15q13.1 | 9266 | 3 | 2.15E−12 | 56.85 | 2.14941E−12 | 1.50E−08 | 2.81E−05 | 4 | 2 |
| [symbol] | [symbol] | [symbol] | [symbol] | [symbol] | [symbol] | [symbol] | [symbol] | [symbol] | [symbol] |
| 22q11.22 | 359 | 2 | 4.50E−32 | 147.32 | 4.50117E−32 | 1.58E−21 | 2.70E−11 | 64 | 1 |
| [symbol] | [symbol] | [symbol] | [symbol] | [symbol] | [symbol] | [symbol] | [symbol] | [symbol] | [symbol] |
| 22q11.22 | 1 | 1 | 6.77E−32 | 40.93 | 6.76528E−32 | 1.94E−19 | 1.16E−11 | 53 | 0 |
| [symbol] | [symbol] | [symbol] | [symbol] | [symbol] | [symbol] | [symbol] | [symbol] | [symbol] | [symbol] |
| 22q11.22 | 5387 | 3 | 3.16E−32 | 77.17 | 3.16442E−32 | 1.75E−20 | 1.20E−13 | 59 | 0 |
| [symbol] | [symbol] | [symbol] | [symbol] | [symbol] | [symbol] | [symbol] | [symbol] | [symbol] | [symbol] |
| 22q11.22 | 18133 | 2 | 1.04E−30 | 73.65 | 1.04248E−30 | 2.41E−58 | 2.32E−10 | 64 | 2 |

| delPval | delOR | dupCases | dupControls | dupPval | dupOR | cnvCases | cnvControls | cnvPval | cnvOR | geneHit |
|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] |
| 1.46E−18 | 1.58 | 48 | 313 | 1.00E+00 | 3.36E−01 | 824 | 1528 | 1.75E−07 | 1.29 | OR4S2 |
| [symbol] | [symbol] | [symbol] | [symbol] | [symbol] | [symbol] | [symbol] | [symbol] | [symbol] | [symbol] | [symbol] |
| 1.73E−01 | 3.39 | 26 | 1 | 8.28E−13 | 5.91E+01 | 29 | 3 | 2.15E−12 | 22.00 | MKRN3 |
| [symbol] | [symbol] | [symbol] | [symbol] | [symbol] | [symbol] | [symbol] | [symbol] | [symbol] | [symbol] | [symbol] |
| 1.73E−01 | 3.39 | 25 | 1 | 2.61E−12 | 5.68E+01 | 28 | 3 | 6.39E−12 | 21.24 | NDN |
| [symbol] | [symbol] | [symbol] | [symbol] | [symbol] | [symbol] | [symbol] | [symbol] | [symbol] | [symbol] | [symbol] |
| 1.73E−01 | 3.39 | 25 | 1 | 2.61E−12 | 5.68E+01 | 28 | 3 | 6.39E−12 | 21.24 | PAR-SN SNURF |
| | | | | | | | | | | PAR5 SNRPN |
| | | | | | | | | | | SNORD 107-109 |
| [symbol] | [symbol] | [symbol] | [symbol] | [symbol] | [symbol] | [symbol] | [symbol] | [symbol] | [symbol] | [symbol] |
| 6.67E−01 | 1.13 | 25 | 1 | 2.61E−12 | 5.68E+01 | 26 | 3 | 5.59E−11 | 19.71 | SNORD109 115 |
| | | | | | | | | | | HBII-52 |
| [symbol] | [symbol] | [symbol] | [symbol] | [symbol] | [symbol] | [symbol] | [symbol] | [symbol] | [symbol] | [symbol] |
| 1.73E−01 | 3.39 | 25 | 1 | 2.61E−12 | 5.68E+01 | 28 | 3 | 6.39E−12 | 21.24 | ATP10A |
| [symbol] | [symbol] | [symbol] | [symbol] | [symbol] | [symbol] | [symbol] | [symbol] | [symbol] | [symbol] | [symbol] |
| 1.73E−01 | 3.39 | 25 | 1 | 2.61E−12 | 5.68E+01 | 28 | 3 | 6.39E−12 | 21.24 | GABRA5 |
| | | | | | | | | | | GABRG3 |
| [symbol] | [symbol] | [symbol] | [symbol] | [symbol] | [symbol] | [symbol] | [symbol] | [symbol] | [symbol] | [symbol] |
| 7.62E−02 | 4.52 | 25 | 1 | 2.61E−12 | 5.68E+01 | 29 | 3 | 2.15E−12 | 22.00 | OCA2 |
| [symbol] | [symbol] | [symbol] | [symbol] | [symbol] | [symbol] | [symbol] | [symbol] | [symbol] | [symbol] | [symbol] |
| 4.50E−32 | 147.32 | 12 | 38 | 8.84E−01 | 7.12E−01 | 76 | 39 | 4.43E−15 | 4.48 | ZNF280B |
| [symbol] | [symbol] | [symbol] | [symbol] | [symbol] | [symbol] | [symbol] | [symbol] | [symbol] | [symbol] | [symbol] |
| 4.95E−28 | None | 18 | 4 | 1.06E−06 | 1.02E+01 | 71 | 4 | 6.77E−32 | 40.93 | PRAME |
| [symbol] | [symbol] | [symbol] | [symbol] | [symbol] | [symbol] | [symbol] | [symbol] | [symbol] | [symbol] | [symbol] |
| 3.86E−31 | None | 8 | 2 | 1.87E−03 | 9.05E+00 | 67 | 2 | 3.16E−32 | 77.17 | LOC648691 |
| [symbol] | [symbol] | [symbol] | [symbol] | [symbol] | [symbol] | [symbol] | [symbol] | [symbol] | [symbol] | [symbol] |
| 1.04E−30 | 73.65 | 83 | 151 | 6.49E−02 | 1.25E+00 | 147 | 153 | 1.43E−11 | 2.22 | MIR650 |

[symbol]

indicates data missing or illegible when filed

### Example II

### Screening Assays for Identifying Efficacious Therapeutics for the Treatment of Autism and ASD

The information herein above can be applied clinically to patients for diagnosing an increased susceptibility for developing autism or autism spectrum disorder and therapeutic intervention. A preferred embodiment of the invention comprises clinical application of the information described herein to a patient. Diagnostic compositions, including microarrays, and methods can be designed to identify the genetic alterations described herein in nucleic acids from a patient to assess susceptibility for developing autism or ASD. This can occur after a patient arrives in the clinic; the patient has blood drawn, and using the diagnostic methods described herein, a clinician can detect a CNV as described in Example I and set forth in Table II. The information obtained from the patient sample, which can optionally be amplified prior to assessment, will be used to diagnose a patient with an increased or decreased susceptibility for developing autism or ASD. Kits for performing the diagnostic method of the invention are also provided herein. Such kits comprise a microarray comprising nucleic acids containing at least one of the CNV/SNPs provided herein in and the necessary reagents for assessing the patient samples as described above.

The identity of autism/ASD involved genes and the patient results will indicate which variants are present, and will identify those that possess an altered risk for developing ASD. The information provided herein allows for therapeutic intervention at earlier times in disease progression than previously possible. Also as described herein above, the CNV containing genes described herein provide novel targets for the development of new therapeutic agents efficacious for the treatment of this neurological disease.

The information provided herein can also be employed in a test and treat approach. Although relatively common, the ASDs are still relatively under diagnosed, especially in rural areas where community pediatricians may not be as knowledgeable about the deluge of research that continues to define these disorders and their treatment. By and large, the mainstay of treatment for the ASDs remains behavioral therapy. There are many different types of behavioral therapies that specifically cater to the diverse phenotypic manifestations of the ASDs, and in all cases earlier intervention is correlated with better results. Therefore, early diagnosis of particular ASD subtypes is crucial to early behavioral intervention (and psychopharmacological management in extreme cases) and maximizing a child's potential and quality of life.

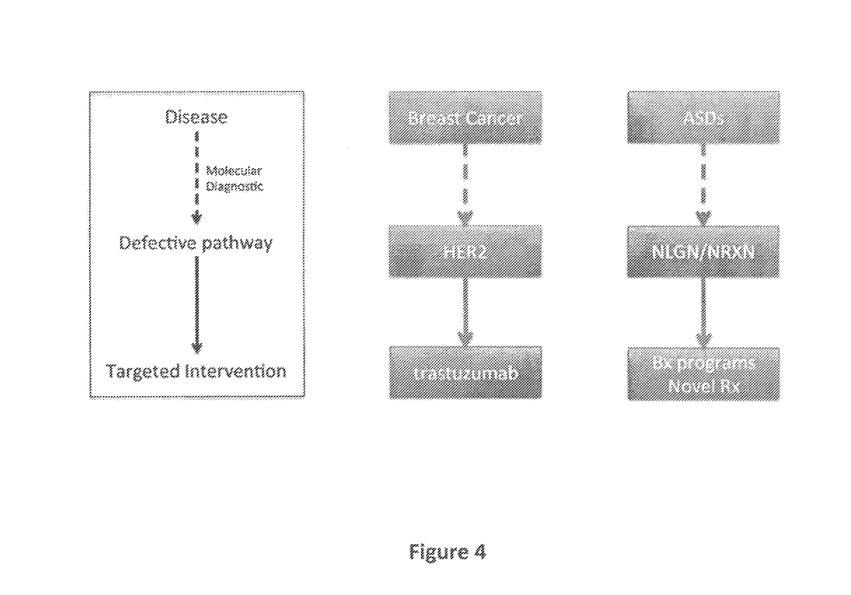

From a pharmacogenomics perspective using breast cancer as an analogy, trastuzumab, a monoclonal antibody that targets HER2 expressing cancer cells, has revolutionized the treatment for breast cancer, and it is perhaps the best-known example of a successful personalized therapeutic (FIG. 4). Before trastuzumab and other personalized therapeutics for cancer, patients would have to undergo relatively crude interventions such as radical mastectomy, radiation, and chemotherapy with low efficacy and severe undesirable side effects. When trastuzumab binds to defective HER2 proteins, it prevents the HER2 protein from causing cells in the breast to reproduce uncontrollably which increases the survival of breast cancer patients. Trastuzumab is only effective in HER2 expressing cancer cells so patients must undergo a genetic test to determine whether their cancer will respond to treatment. In fact, this test and treat model for targeted therapy is now the gold standard for breast cancer treatment.

Recently, next generation sequencing (NGS) was employed to study the genetic etiology of the ASD in sporadic families by analyzing the sequenced exomes of 20 parent-child trios, (O'Roak et al. 2011). These studies revealed four attractive candidate genes (FOXP1, GRIN2B, SCN1A and LAMC3) involved in neurotransmission which harbored functional de-novo mutations in sporadic families with ASDs. The notion that as few as 60 exomes could facilitate the identification of four rare plausible functional mutations underlying the ASDs suggests that as NGS of larger numbers of samples becomes more commonplace, the genomic landscape of rare mutations underlying ASDs will expand considerably to the benefit of clinicians and patients alike. Just as a better understanding of the molecular genetics of cancer cells has revolutionized the treatment of breast and other cancer treatments, improved resolution to identify rare mutations underlying ASDs will facilitate the development of molecular tests with better diagnostic yields that will be able to aid clinicians in diagnosing the ASDs and their particular genetic sub-types.

With better molecular diagnostics that dissect the sporadic genetic mutations underlying the ASDs, the personalized approach to treating patients' specific molecular defects in becomes a reality. This type of cutting-edge pharmacogenomics approach to the ASDs will facilitate the development of a test and treat model for drugs that target genetically defined responder populations just as trastuzumab does for HER2+breast cancer patients (FIG. 4). Indeed SSRIs have already proved efficacious for rare cases of ASDs where the serotonin system is defective, and work is apace to define analogous treatments for ASDs due to malfunctioning NLGN/NRXN pathways, neuronal cell adhesion pathways, glutamatergic receptor pathways, and a host of other neurophysiological pathways and networks that have been shown to be defective in sporadic cases.

Having already implicated the GABAR-A pathway by CNV analysis as described above in Example 1, we have gone on to identify a host of drug candidates that act on this signalling pathway to potentially rescue underlying neurogenetic defects in patients with the ASDs (Tables 3 and 4). Topiramate is one such candidate that acts as an agonist at the GABAR-A pathway. Just as we did for the GABAR-A pathway itself in example 1, we defined the first-degree interactome of topiramate itself, and a found that ASD cases are significantly enriched for pathway defects in cases when compared with neurologically normal controls. About 20.7% of cases harbor genetic pathway defects vs 8.3% of controls, a statistically significant difference (P<=1.5×0⁴⁴ OR=2. 9) that supports our hypothesis that topiramate itself may be effective in treating patients with ASDs that harbor genetic defects in the GABAR-A pathway (FIG. 4).

The rational approach to personalized drug design as described herein should both restore normal neurophysiology in patients with ASDs by rescuing specific disrupted genetic pathways and avoid exposing them to drugs that will precipitate adverse side effects. Given the immense clinical and genetic heterogeneity of the ASDs, early tailored psychopharmacgenomic intervention as we have outlined here in combination with comprehensive behavioral programs should improve the prognosis and the outlook for patients that suffer from these burdensome diseases.

TABLE 3

| Active Ingredient | Phase | Description | Indication | MOA |
|---|---|---|---|---|
| | II | BL-1020 is an orally available small | Schizophrenia | Dopamine Receptor |
| | | molecule that acts as a dopamine receptor | | Antagonist; Gamma- |
| | | antagonist and GABA A receptor agonist. | | Aminobutyric Acid Type A |
| | | It is a novel antipsychotic agent, which | | (GABAA) Receptor Agonist |
| | | blocks hyperactivity of the | | |
| | | neurotransmitter dopamine, | | |
| | | simultaneously raising the levels of the | | |
| | | neurotransmitter GABA. BL-1020 is | | |
| | | being developed as an oral formulation | | |
| | | for the treatment of schizophrenia. | | |
| amobarbital | M | Amylobarbitone belongs to barbiturates | Insomnia | Gamma-Aminobutyric Acid |
| | | that act to increase the actions of GABA | | Type A (GABAA) Receptor |
| | | which cause decreased nerve activity in | | Agonist |
| | | the brain it is developed to produce | | |
| | | sleepiness. It is indicated in the treatment | | |
| | | of insomnia and available as tablets | | |
| | | (10 mg, 25 mg, 50 mg). | | |
| butabarbital sodium | M | Butisol Sodium contains butabarbital | Insomnia | Gamma-Aminobutyric Acid |
| | | sodium as active ingredient. Butabarbital | | Type A (GABAA) Receptor |
| | | slows the brain and makes the person | | Agonist |
| | | calm and sleepy. Butisol Sodium is | | |
| | | indicated as a daytime sedative when mild | | |
| | | sedation is required for relief of | | |
| | | symptoms of anxiety or tension resulting | | |
| | | from emotional, physical, or situational | | |
| | | stress. It is available as tablets (30 mg, | | |
| | | 50 mg) and elixir (30 mg/5 ml) for oral | | |
| | | administration. Note: This product is | | |
| | | added upon the acquisition of MedPointe | | |
| | | Healthcare Inc. | | |
| clomethiazole | M | Heminevrin contains clomethiazole, a | Insomnia | Gamma-Aminobutyric Acid |
| | | sedative and hypnotic that acts through | | Type A (GABAA) Receptor |
| | | the GABA receptor as an agonist and | | Agonist |
| | | enhances the neurotransmitter GABA. | | |
| | | Heminevrin is indicated for the treatment | | |
| | | of insomnia and is available in the form | | |
| | | of capsules (300 mg). | | |
| cyclobarbital calcium | M | Cyclobarbitone contains cyclobarbital | Epilepsy | Gamma-Aminobutyric Acid |
| | | calcium, a hypnotic and sedative acts on | | Type A (GABAA) Receptor |
| | | GABA-A receptors, increasing synaptic | | Agonist |
| | | inhibition which controls CNS arousal. It | | |
| | | is indicated for the treatment of epilepsy | | |
| | | and is available as tablets (50 mg, 100 mg, | | |
| | | 200 mg). | | |
| eszopiclone* | M | Eszopiclone, a nonbenzodiazepine | Insomnia | Gamma-Aminobutyric Acid |
| | | hypnotic agent is a pyrrolopyrazine | | Type A (GABAA) Receptor |
| | | derivative of the cyclopyrrolone class, | | Agonist |
| | | acts by interacting with GABA receptor | | |
| | | complexes at binding domains located | | |
| | | close to or allosterically coupled to | | |
| | | benzodiazepine receptors. S-Clon is used | | |
| | | for the treatment of insomnia and is | | |
| | | available as tablets. | | |
| etifoxine hydrochloride | M | Stresam contains etifoxine hydrochloride | Anxiety | Gamma-Aminobutyric Acid |
| | | a non benzodiazepine anxiolytic agent | | Type A (GABAA) Receptor |
| | | which acts by potentiating GABA (A) | | Agonist |
| | | receptor function by a direct allosteric | | |
| | | effect and by an indirect mechanism | | |
| | | involving the activation of peripheral | | |
| | | benzodiazepine receptors (PBRs). | | |
| | | Stresam is indicated for the treatment of | | |
| | | anxiety and is available as capsules | | |
| | | (50 mg). | | |
| meprobamate | M | Meprobamate is a propanediol anxiolytic | Anxiety | Gamma-Aminobutync Acid |
| | | agent which acts on the central nervous | | Type A (GABAA) Receptor |
| | | system causing muscle relaxation. | | Agonist |
| | | Meprobamate is indicated for the | | |
| | | treatment of anxiety and is available as | | |
| | | tablets (200 mg, 400 mg). | | |
| moxidectin | III | Moxidectin is a potent broad-spectrum | Infection | Gamma-Aminobutyric Acid |
| | | endectocide of the macrocyclic lactone | (Onchocerciasis) | Type A (GABAA) Receptor |
| | | (macrolide) antimicrobial class. The | | Agonist |
| | | macrocyclic lactones consist of two | | |
| | | closely related chemical groups, | | |
| | | avermectins and milbemycins. | | |
| | | Moxidectin is being developed as oral | | |
| | | tablets for the treatment of onchocerciasis | | |
| | | (River Blindness). Note: This product is | | |
| | | added upon the acquisition of Wyeth. | | |
| pagoclone | III | Pagoclone, a novel member of the | Stuttering | Gamma-Aminobutyric Acid |
| | | cyclopyrrolone class of compounds, | | Type A (GABAA) Receptor |
| | | which acts as a gamma amino butyric acid | | Agonist |
| | | (GABA) receptor modulator. It increases | | |
| | | the action of GABA, thus reducing excess | | |
| | | neuronal activity. IP456 is being | | |
| | | developed as an oral formulation for the | | |
| | | treatment of persistent developmental | | |
| | | stuttering. Note: This product is added | | |
| | | upon acquisition of Indevus | | |
| | | Pharmaceuticals Inc. | | |
| phenobarbital | M | Phenobarbital is a barbiturate which acts | Epilepsy | Gamma-Aminobutyric Acid |
| | | on GABA A receptors by increasing | | Type A (GABAA) Receptor |
| | | synaptic inhibition that results in | | Agonist |
| | | elevating seizure threshold and reducing | | |
| | | the spread of seizure activity from a | | |
| | | seizure focus. Phenobarbital is indicated | | |
| | | for the treatment of epilepsy. It is | | |
| | | available as tablets (15 mg, 60 mg, 100 mg) | | |
| | | and injection (200 mg/ml). | | |
| phenobarbital | M | Phenobarbitone is a barbituric acid | Insomnia | Gamma-Aminobutyric Acid |
| | | derivative, a nonselective central nervous | | Type A (GABAA) Receptor |
| | | system depressant which acts on GABAA | | Agonist |
| | | receptors, increasing synaptic inhibition. | | |
| | | This has the effect of elevating seizure | | |
| | | threshold and reducing the spread of | | |
| | | seizure activity from a seizure focus. | | |
| | | Phenobarbitone may also inhibit calcium | | |
| | | channels, resulting in a decrease in | | |
| | | excitatory transmitter release. The | | |
| | | sedative-hypnotic effects of phenobarbital | | |
| | | are likely the result of its effect on the | | |
| | | polysynaptic midbrain reticular | | |
| | | formation, which controls CNS arousal. | | |
| | | Phenobarbitone is indicated for the | | |
| | | treatment of insomnia and is available as | | |
| | | tablets (15 mg, 30 mg, 60 mg, and 100 mg). | | |
| phenobarbital sodium | M | Phenobarbital sodium is a longest-acting | Epilepsy | Gamma-Aminobutyric Acid |
| | | barbiturate with anticonvulsant and | | Type A (GABAA) Receptor |
| | | sedative-hypnotic properties. It acts on | | Agonist |
| | | GABAA receptors, increasing synaptic | | |
| | | inhibition which results in elevating | | |
| | | seizure threshold and reducing the spread | | |
| | | of seizure activity from a seizure focus. | | |
| | | Phenobarbital may also inhibit calcium | | |
| | | channels, resulting in a decrease in | | |
| | | excitatory transmitter release. It is | | |
| | | indicated for the treatment of epilepsy and | | |
| | | available as intramuscular and | | |
| | | intravenous injection (65 mg/ml and | | |
| | | 130 mg/ml). | | |
| phenprobamate | M | Phenprobamate is a centrally acting | Spasm | Gamma-Aminobutyric Acid |
| | | skeletal muscle relaxant. It acts by | | Type A (GABAA) Receptor |
| | | interrupting neuronal communication in | | Agonist |
| | | the reticular formation and spinal cord, | | |
| | | causing sedation and altered perception of | | |
| | | pain. Phenprobamate is indicated for the | | |
| | | treatment of musculoskeletal and muscle | | |
| | | spasms and is available as tablets (0.2 g). | | |
| primidone | M | Primidone is an anticonvulsant agent that | Epilepsy | Gamma-Aminobutyric Acid |
| | | is structurally related to barbiturates. It is | | Type A (GABAA) Receptor |
| | | a gamma-aminobutyric acid type (GABA) | | Agonist |
| | | receptor agonist that increases the | | |
| | | synaptic inhibition, elevating seizure | | |
| | | threshold and reducing the spread of | | |
| | | seizure activity from a seizure focus. | | |
| | | Primidone is indicated alone or | | |
| | | concomitantly with other anticonvulsants | | |
| | | for the control of grand mal, psychomotor | | |
| | | and focal epileptic seizures. It is available | | |
| | | as tablets (50 mg and 250 mg). | | |
| topiramate | M | Adco-Topiramate contains topiramate, | Epilepsy | Alpha-Amino-3-Hydroxy-5- |
| | | a sulfamate-substituted | | Methyl-4- |
| | | monosaccbaride and an anticonvulsant | | Isoxazolepropionic Acid |
| | | agent. It blocks the voltage dependent | | (AMPA) Receptor |
| | | sodium channels, augments the activity | | Antagonist; Gamma- |
| | | of the neurotransmitter gamma amino | | Aminobutyric Acid Type A |
| | | butyrate at some subtypes of the | | (GABAA) Receptor |
| | | GABA-A receptor, antagonizes the | | Agonist; Sodium Channel |
| | | AMPA/kainate subtype of the | | Blocker |
| | | glutamate receptor and inhibits the | | |
| | | carbonic anhydrase enzyme, | | |
| | | particularly isozymes II and IV. Adco- | | |
| | | Topiramate is indicated for the | | |
| | | treatment of partial onset seizures, | | |
| | | primary generalized tonic clonic | | |
| | | seizures and seizures associated with | | |
| | | lennox-gastaut syndrome. It is available | | |
| | | as tablets (25 mg, 50 mg, 100 mg, 200 mg). | | |
| zaleplon* | M | Zaleplon is a short-acting, non- | Insomnia | Gamma-Aminobutyric Acid |
| | | benzodiazepine sedative-hypnotic. It acts | | Type A (GABAA) Receptor |
| | | as an agonist at type 1 benzodiazepine | | Agonist |
| | | (BZ1 or omega1) receptors on the | | |
| | | GABA-A/chloride-ion channel complex | | |
| | | within the CNS. Zaleplon is indicated for | | |
| | | the treatment of insomnia and is available | | |
| | | as capsules (5 mg and 10 mg). | | |
| zolpidem* | M | Zolpidem is a non-benzodiazepine | Insomnia | Gamma-Aminobutyric Acid |
| | | hypnotic of the imidazopyridine class. It | | Type A (GABAA) Receptor |
| | | acts by increasing the activity of GABA, | | Agonist |
| | | there by reducing the functioning of | | |
| | | certain areas of the brain. It results in | | |
| | | sleepiness, decrease in anxiety and | | |
| | | relaxation of muscles. Zolpidem is | | |
| | | indicated for the treatment of insomnia | | |
| | | and is available as film coated tablets | | |
| | | (5 mg, 10 mg). | | |
| zolpidem tartrate* | M | Zolfresh contains zolpidem tartrate, a | Insomnia | Gamma-Aminobutyric Acid |
| | | nonbenzodiazepine sedative-hypnotic, | | Type A (GABAA) Receptor |
| | | which belongs to the imidazopyridine | | Agonist |
| | | class of drugs. It is a gamma-amino-n- | | |
| | | butyric acid type A (GABA-A) receptor | | |
| | | agonist which works by binding | | |
| | | preferentially to the omega-1 (BZ-1) | | |
| | | receptor subtype of the GABAA receptor | | |
| | | complex. Zolfresh is indicated for the | | |
| | | short-term treatment of insomnia and is | | |
| | | available as film coated tablets (5 mg, | | |
| | | 10 mg). | | |
| zopiclone* | M | Zopimed contains zopiclone, a | | Gamma-Aminobutyric Acid |
| | | nonbenzodiazepine hypnotic of the | | Type A (GABAA) Receptor |
| | | pyrazolopyrimidine class. It binds | | Agonist |
| | | selectively to the brain alpha subunit of | | |
| | | the gamma aminobutyric acid (GABA A) | | |
| | | omega 1 receptor and exerts its action by | | |
| | | modulation of the GABA benzodiazepine | | |
| | | receptor chloride channel macromolecular | | |
| | | complex. Zopimed is indicated for the | | |
| | | short-term treatment of insomnia in adults | | |
| | | and is available as tablets (7.5 mg). | | |

GABA-A agonist drugs
*structurally not like benzodiazepine, but have similar effects.

TABLE 4

| | Active | | | |
|---|---|---|---|---|
| Product | Ingredient | Phase | Indication | MOA |
| NGD912 | — | F | Anxiety | Gamma-Aminobutyric Acid (GABA) Receptor Agonist |
| NGD913 | — | F | Anxiety | Gamma-Aminobutyric Acid (GABA) Receptor Agonist |
| Gaboxadol | THIP | D | Insomnia | GABA-A recceptor agonist |
| CPP109 | vigabatrin | I | Drug Abuse (Addiction | Gamma-Aminobutyric Acid Transaminase (GABA-T) Inhibitor |
| | | | on both Cocaine and | |
| | | | Alcohol) | |
| CPP115 | — | I | Cocaine Addiction | Gamma-Aminobutyric Acid Transaminase (GABA-T) Inhibitor |
| SUN09 | — | I | Neurological Disorders | Gamma-Aminobutyric Acid Type B (GABAB) Receptor Agonist |
| | | | (Muscle Spasticity) | |
| AGI006 | arbaclofen | II | Indigestion (Functional | Gamma-Aminobutyric Acid Type B (GABAB) Receptor Agonist |
| | | | Dyspepsia) | |
| DP-VPA | — | II | Epilepsy | Gamma-Aminobutyric Acid Transaminase (GABA-T) Inhibitor |
| Xyrem | sodium | II | Schizophrenia | Gamma-Aminobutyric Acid Type B (GABAB) Receptor Agonist |
| | oxybate | | | |
| STX209 | arbaclofen | III | Fragile X syndrome | |
| IP456 | pagoclone | III | Stuttering | Gamma-Aminobutyric Acid Type A (GABAA) Receptor Agonist |
| XP19986 | arbaclofen | III | Neurological disorders | Gamma-Aminobutyric Acid Type B (GABAB) Receptor Agonist |
| | placarbil | | | |
| | baclofen | M | Spasm | Gamma-Aminobutyric Acid Type B (GABAB) Receptor Agonist |
| | vigabatrin | M | Epilepsy | Gamma-Aminobutyric Acid Transaminase (GABA-T) Inhibitor |
| Xyrem | sodium | M | Marcolepsy | Gamma-Aminobutyric Acid Type B (GABAB) Receptor Agonist |
| | oxybate | | | |
| Lyrica | Pregabalin | M | Anxiety, | GABA analog |
| | | | epilepsy, Neuralgia, | |
| | | | Diabetic | |
| | Gabapentin | M | Epilepsy | GABA analog |
| Heminevrin | clomethiazole | M | Insomenia in Europe | Gamma-Aminobutyric Acid Type A (GABAA) Receptor Agonist |
| Gabitril | tiagabine | M | Epilepsy | GABA reuptake inhibitor |
| | hydrochloride | | | |

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for detecting a propensity for developing autism or autistic spectrum disorder, the method comprising:
a) providing a plurality of CNVs present in autistic or ASD patients identified using genome wide association studies of large patient cohorts, said CNVs being listed in Table II;
a) obtaining a sample from a patient and isolating nucleic acid therefrom;
b) identifying the presence or absence of at least one deletion containing CNV in a target polynucleotide, wherein if said CNV is present, said patient has an increased risk for developing autism and/or autistic spectrum disorder.

2. A method as claimed in claim 1, wherein the target polynucleotide is amplified prior to detection.

3. The method of claim 1, wherein the step of identifying the presence of said CNV further comprises the step of analyzing said nucleic acid by performing a process selected from the group consisting of detection of specific hybridization, measurement of allele size, restriction fragment length polymorphism analysis, allele-specific hybridization analysis, single base primer extension reaction, and sequencing of an amplified polynucleotide.

4. A method as claimed in claim 1, wherein in the target nucleic acid is DNA.

5. The method of claim 1, wherein polynucleotide comprising said CNV is obtained from an isolated cell of the human subject.

6. A method for identifying agents which alter neuronal signaling and/or morphology, comprising
a) providing cells expressing at least one CNV as claimed in claim 1;
b) providing cells which express the cognate wild type sequences corresponding to the CNV of step a);
c) contacting the cells of steps a) and b) with a test agent; and
d) analyzing whether said agent alters neuronal signaling and/or morphology of cells of step a) relative to those of step b), thereby identifying agents which alter neuronal signaling and morphology.

7. A method of treating autism or ASD in a human subject determined to have at least copy number variation (CNV) associated with an autistic or ASD phenotype, said at least one CNV being selected from the group consisting of CNVs set out in Table 2 via the method of claim 1, the method comprising administering to said human subject a therapeutically effective amount of at least one agent which is known to be efficacious in the signaling pathway adversely affected by the presence of said CNV.

8. The method of claim 7, wherein said CNV containing gene is selected from the group consisting of ATP10A, GABRA5, GABRB3, GABRG3, GGTLC2, HBII-52-45, HBII-52-46, IPW, LOC648691, LOC96610, MAGEL2, MIR650, MKRN3, NCRNA00221, NDN, OCA2, OR4S2, PAR-SN, PAR1, PAR5, POM121L1P, PRAME, SNORD107, SNORD108, SNORD109A, SNORD109B, SNORD115-11, SNORD115-29, SNORD115-36, SNORD115-43, SNORD115-44, SNORD115-48, SNORD64, SNRPN, SNURF, UBE3A, ZNF280A, ZNF280B.

9. The method of claim 7, wherein said agent alters GABA signaling and is listed in Table 3 or Table 4.

10. The method of claim 9, wherein said gene is selected from the group consisting of GABRA5, GABRA3, GABRB3 and said agent is topiramide.

11. A method for detecting a propensity for developing autism or autistic spectrum disorder, the method comprising:
a) obtaining a sample from a patient and isolating nucleic acid therefrom;
b) amplifying the nucleic acid of step a);
c) contacting a microarray comprising nucleic acids of known sequence with the amplified nucleic acids of step b) and identifying hybridizing nucleic acids if any, thereby identifying the presence or absence of at least one deletion containing CNV in a target polynucleotide, said CNV being listed in Table II; wherein if said CNV is present, said patient has an increased risk for developing autism and/or autistic spectrum disorder.

12. The method of claim 11, further comprising isolation of said CNV containing nucleic acid.

13. The method of claim 11, further comprising administration of an effective amount of an agent useful to alleviate ASD symptoms.

14. A kit for practicing the method of claim 1.

15. A kit for practicing the method of claim 11.
